(19) United States
(12) Patent Application Publication
(10) Pub. No.: US 2007/0207947 A1
(43) Pub. Date: Sep. 6, 2007

(54) Stabilized Peptides

(75) Inventors: Hans-Georg Frank, Kerkrade (NL); Udo Haberl, Baesweiler (DE); Franzpeter Bracht, Dusseldorf (DE); Andreas Rybka, Gelsenkirchen (DE)

Correspondence Address: JACOBSON HOLMAN PLLC, 400 SEVENTH STREET N.W., SUITE 600, WASHINGTON, DC, 20004, US

(73) Assignee: APLAGEN GMBH, Baesweiler (DE)

(21) Appl. No.: 10/575,864

(22) PCT Filed: Oct. 18, 2004
(86) PCT No.: PCT/EP04/11719

§ 371 Date: May 15, 2007

(30) Foreign Application Priority Data
Oct. 16, 2003 (EP) 03023395.1

(51) Int. Cl. A61K 38/14; A61K 38/12; C07K 7/60

(52) U.S. Cl. 514/8; 530/317; 514/9

(57) ABSTRACT

The present invention therefore presents modules from which helical constraints can be built by very flexible strategies. The peptide bonds involved partially compensate the hydrophobic nature of the disulfide bonds, which are also included into the constraint strategy. Thus, the invention presents solutions, by means of which peptide bonds or closure of disulfide bridges can be used alternatively for closure of the constraint. This offers greater synthetic flexibility. Moreover, the peptide bonds are more hydrophilic than disulfide bridges alone and offer the advantage of better solubility of the product in an aqueous surrounding. It is possible to attach solvation tags like glycosyl moieties, polyethylenglycol or other suitable extensions or appendices to the helical constraint structure. Usually, such a hydrophilic helical constraint structure replaces two hydrophobic amino acid side chains and thus improves pharmacologic properties of the molecule.

### FIELD OF THE INVENTION

A common principle of the structure of many naturally occurring proteins is the presence of helical domains. Usually, such helical parts of proteins contain 20-30 amino acid residues and are a typical element of the secondary structure of proteins. Typical examples of such proteins are cytokines like interleukin 2 (Majewski 1996), interleukin 4 (Gustchina, Zdanov et al. 1995; Gustchina, Zdanov et al. 1997) and interleukin 6 (Somers, Stahl et al. 1997), but others like erythropoietin also contain helical substructures (Sytkowski and Grodberg 1997), which usually participate in the cytokine/receptor interactions. In such proteins helices are frequently assembled around a hydrophobic core, to which hydrophobic amino acid residues project. Around this core they form tertiary structures, for which coiled coil interactions of the type also called “leucine zipper” are typical (Vieth, Kolinski et al. 1994). By this overall constructive principle, such helix-bundle cytokines project their contact surface to the outside of the molecule, while the hydrophobic core forms a stable anchor, around which the helical subdomains cluster. The surface tension of the surrounding aqueous solvent thus is the source of forces, which finally stabilizes the helical parts around the hydrophobic core.

Binding domains of such helix-bundle type cytokines are interesting parts of the molecule and it is tempting to just take the sequence of 20-30 amino acids and to use this part of the molecule alone to bind to the receptor (Theze, Eckenberg et al. 1999). This would aim and would be suitable for both antagonistic or agonistic approaches to minimized cytokines or cytokine antagonists. Moreover, such short stretches can be obtained in a relatively uncomplicated manner by means of classical chemical solid phase peptide synthesis of the Merrifield-type.

### OBJECT OF THE INVENTION

However, reduction to smaller peptides takes away the above mentioned constructive principles and short peptides of 30 amino acids are only partially helical in aqueous solutions (Theze, Eckenberg et al. 1999). The hydrophobic side chains, which projected to the former core part of the complete cytokine are now projecting into the aqueous surrounding, which is a destabilizing factor for the helix and induces a tendency of such molecules to aggregate in irregular clusters. Such clusters might have some residual activity, but sometimes this is just due to irregular ligand/receptor interactions, which are different from the natural type of interactions (Theze, Eckenberg et al. 1999; Eckenberg, Rose et al. 2000). The helical structure, which is needed to bind to the receptor molecules is thus not stable in water. Frequently, the overall helical content, measured by circular dichroism is below 50%. This does not even mean that 50% of the molecules are completely helical, but indicates that the average over all helical interactions is 50%. The actual concentration of completely helical molecules which are needed for adequate binding, is not known and certainly much smaller than 50% of the apparent molar concentration. For all these reasons, the binding constants of small peptides to a given receptor usually do neither qualitatively nor quantitatively match the cytokine receptor interactions although they might harbour a sequence-identical subpart of the molecule.

For the isolation of helical structures from proteins, e.g. cytokines, one has to solve the problem of stabilisation of helices by means of other methods than assembly around a large hydrophobic core.

Several approaches have been reported for stabilizing alpha-helical peptides. Stabilization can be achieved by addition of trifluorethanol or hexafluorisopropanol (Sung and Wu 1996). A variety of non-covalent side chain constraints have been reported including incorporation of metal chelates, salt bridges, and hydrophobic interactions. However, the non-covalent strategies suffer from a number of disadvantages:

Solvents like trifluoroethanol or hexafluorisopropanol increase helical content, but can not be used in pharmaceutical preparations and are certainly not present in sufficient concentrations. in vivo.

Metal chelates and salt bridges induce very large highly polar groups, which—in case of small peptides—are likely to negatively influence the binding to the receptor as well as pharmacokinetic properties of a given molecule. In case of metal chelates only non-toxic metals can be used for pharmaceutically relevant preparations.

Hydrophobic interactions are difficult to control. Irregular aggregation and undesired intermolecular interactions usually form problems, which induce a great loss in active substance being available after a complex synthesis and preparation protocol. Sometimes, only the use of strong detergents and/or organic solvents is able to control aggregation thoroughly. Again, such preparations are difficult to apply to the intended pharmaceutical targets.

Thus, one of the most successful strategies is the stabilization of alpha-helical peptides by covalent bridges which connect the side chains of two appropriately located amino acids. Appropriately implies that the side chains of these amino acids do not participate in the intended binding sites. Moreover, they should stabilize two helical turns, which means a step of 7 amino acids in the sequence. Bridges which connect two side chains at positions i and i+7 can stabilize the helix with little perturbation on helix conformation. Once the helical conformation is enforced by such a construct, the overall helical content improves strongly and the total helical conformation of the peptide becomes kinetically favoured. The constraints of peptides by lactamization (Huston, Houston et al. 1995), amides (Braisted, Judice et al. 1997; Phelan, Skelton et al. 1997; Braisted, Judice et al. 1998) or disulfide bonds (Jackson, King et al. 1991) have been described.

However, all these strategies suffer from the disadvantage, that the synthetic strategy has to be designed such that the closure of the constraint structure of the side chain is possible. In case of disulfide bonds, this becomes difficult as soon as other disulfide bridges have to be closed.

### SUMMARY OF THE INVENTION

Surprisingly, the problem of the invention is solved by peptidic compounds, pharmaceutical preperations, antibodies, compounds as building blocks and synthesis methods of any of claims 1 to 36.

The present invention therefore presents modules from which helical constraints can be built by very flexible strategies. The peptide bonds involved partially compensate the hydrophobic nature of the disulfide bonds, which are also included into the constraint strategy. Thus, the invention presents solutions, by means of which amide bonds or closure of disulfide bridges can be used alternatively for closure of the constraint. This offers greater synthetic flexibility. Moreover, the amide bonds are more hydrophilic than disulfide bridges alone and offer the advantage of better solubility of the product in an aqueous surrounding. Another important advantage of the peptide bonds is the stabilization of the bridge structure by supporting pillars, as shown in the examples below.

This new combination of amide bonds and disulfide bonds has clear advantages over the application of one of these two bond types alone. Especially the disulfide bond is easy to form. One of the intended purposes of the amide bond in the bridge is the stabilization of the bridge strucure, because the amide bond can interact with other amino acid side chains under the bridge which act as supporting pillars (see examples 1 to 3). This stabilization by supporting pillars is not only active in the final (ring-closed) structure, but also before the ring closure, which leads to higher yields in the synthesis of the correctly folded cyclic structure.

Thus the combination of amide bonds and disulfide bonds achieves a new degree of efficiency and provides advantages for synthesis as well as for structure stabilization.

It is also possible to attach solvation tags like glycosyl moieties, polyethylenglycol or other suitable extensions or appendices to the helical constraint structure. Usually, such a hydrophilic helical constraint structure replaces two hydrophobic amino acid side chains and thus improves pharmacologic properties of the molecule.

### DETAILED DESCRIPTION OF THE INVENTION

In general, the invention provides structures which can be adapted to almost every synthetic problem during the synthesis of helically stabilized peptides. Structurally, the bridges, which are constructed alongside the sequence of the peptide, comprise a flexible covalent backbone with at least one amide bond and one disulfide bond. Closure of the bridge by the disulfide bond will e. g. be a good way of formation of the bridge. But if necessary, the bridge can be closed e.g. by on-resin closure of one peptide bond, while the disulfide bridge was already introduced as a ready to use building block. The skilled person knows other possible ways or is able to find other possible ways for performing the invention after reading and understanding the present description of the invention.

In a preferred embodiment of the invention, the amide-bond containing building blocks which form the bridge structures are made by solid phase synthesis. The peptide chemist is familiar with these methods. Thus one of the advantages of these building blocks is that they are synthetically easily available for peptide chemists.

Below, a series of six general formulas will present the whole range of the invention. The invention encompasses helical constrained peptides represented by formula (1) to (7).

Formula (1) represents a compound 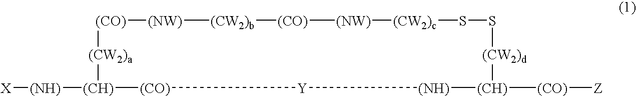
wherein X is hydrogen or any amino acid or any peptide, Y is any amino acid sequence consisting of six amino acids, Z is hydroxyl or any amino acid or any peptide, a, b, c and d are independently selected from the integers 1 to 3, provided that a+b+c+d is any integer in the range from 5 to 9; at each independent position of W, W can be freely chosen from hydrogen, a hydroxyl-, carboxyl- or amino group, an alkyl moiety with at least one hydroxyl-, carboxyl- or amino group, a polyethyleneglycol moiety, or a naturally occurring or artificial sugar molecule, and the peptides can consist of natural and/or unnatural D- and/or L-amino acids. Examples 1 to 4 demonstrate the application of this formula.

Formula (2) represents a compound 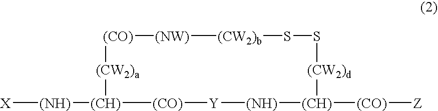
wherein X is hydrogen or any amino acid or any peptide, Y is any amino acid sequence consisting of six amino acids, Z is hydroxyl or any amino acid or any peptide, a, b and d are independently selected from the integers 1 to 5, provided that a+b+d is any integer in the range from 7 to 11; at each independent position of W, W can be freely chosen from hydrogen, a hydroxyl-, carboxyl- or amino group, an alkyl moiety with at least one hydroxyl-, carboxyl- or amino group, a polyethyleneglycol moiety, or a naturally occurring or artificial sugar molecule, and the peptides can consist of natural and/or unnatural D- and/or L-amino acids. Example 5 illustrates this formula.

Formula (3) represents a compound 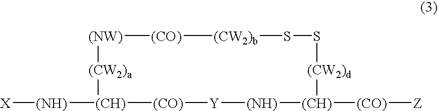
wherein X is hydrogen or any amino acid or any peptide, Y is any amino acid sequence consisting of six amino acids, Z is hydroxyl or any amino acid or any peptide, a, b and d are independently selected from the integers 1 to 5, provided that a+b+d is any integer in the range from 7 to 11; at each independent position of W, W can be freely chosen from hydrogen, a hydroxyl-, carboxyl- or amino group, an alkyl moiety with at least one hydroxyl-, carboxyl- or amino group, a polyethyleneglycol moiety, or a naturally occurring or artificial sugar molecule, and the peptides can consist of natural and/or unnatural D- and/or L-amino acids. Example 6 illustrates this formula.

Formula (4) represents a compound 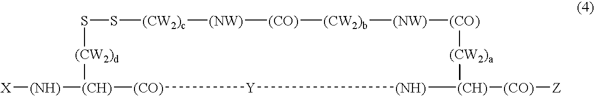
wherein X is hydrogen or any amino acid or any peptide or any compound represented by formula (1) to (2), Y is any amino acid sequence consisting of six amino acids, Z is hydroxyl or any amino acid or any peptide or any compound represented by formula (1) to (6), a, b, c and d are independently selected from the integers 1 to 3, provided that a+b+c+d is any integer in the range from 5 to 9 and the peptides can consist of natural and/or unnatural D- and/or L-amino acids; at each independent position of W, W can be freely chosen from hydrogen, a hydroxyl-, carboxyl- or amino group, an alkyl moiety with at least one hydroxyl-, carboxyl- or amino group, a polyethyleneglycol moiety, or a naturally occurring or artificial sugar molecule, and the peptides can consist of natural and/or unnatural D- and/or L-amino acids. Example 7 illustrates the application of this formula.

Formula (5) represents a compound 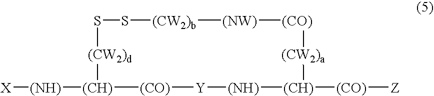
wherein X is hydrogen or any amino acid or any peptide or any compound represented by formula (1) to (6), Y is any amino acid sequence consisting of six amino acids, Z is hydroxyl or any amino acid or any peptide or any compound represented by formula (1) to (6), a, b and d are independently selected from the integers 1 to 5, provided that a+b+d is any integer in the range from 7 to 11 and the peptides can consist of natural and/or unnatural D- and/or L-amino acids; at each independent position of W, W can be freely chosen from hydrogen, a hydroxyl-, carboxyl- or amino group, an alkyl moiety with at least one hydroxyl-, carboxyl- or amino group, a polyethyleneglycol moiety, or a naturally occurring or artificial sugar molecule, and the peptides can consist of natural and/or unnatural D- and/or L-amino acids. Example 8 illustrates this type of formula.

Formula (6) represents a compound 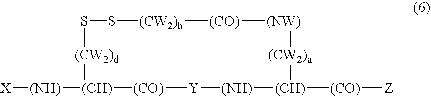
wherein X is hydrogen or any amino acid or any peptide or any compound represented by formula (1) to (6), Y is any amino acid sequence consisting of six amino acids, Z is hydroxyl or any amino acid or any peptide or any compound represented by formula (1) to (6), a, b and d are independently selected from the integers 1 to 5, provided that a+b+d is any integer in the range from 7 to 11 and the peptides can consist of natural and/or unnatural D- and/or L-amino acids; at each independent position of W, W can be freely chosen from hydrogen, a hydroxyl-, carboxyl- or amino group, an alkyl moiety with at least one hydroxyl-, carboxyl- or amino group, a polyethyleneglycol moiety, or a naturally occurring or artificial sugar molecule, and the peptides can consist of natural and/or unnatural D- and/or L-amino acids. Examples 9 and 10 illustrate the application of this formula.

Amino acids described in this invention can be of the naturally occuring L stereoisomer form as well as the enantiomeric D form. The one-letter code refers to the accepted standard polypeptide nomenclature, but can mean alternatively a D- or L-amino acid:

Code amino acids

A L-Alanine or D-Alanine
V L-Valine or D-Valine
L L-Leucine or D-Leucine
I L-Isoleucine or D-Isoleucine
M L-Methionine or D-Methionine
F L-Phenylalanine or D-Phenylalanine
Y L-Tyrosine or D-Tyrosine
W L-Tryptophan or D-Tryptophan
H L-Histidine or D-Histidine
S L-Serine or D-Serine
T L-Threonine or D-Threonine
C L-Cysteine or D-Cysteine
N L-Asparagine or D-Asparagine
Q L-Glutamine or D-Glutamine
D L-Aspartic acid or D-Aspartic acid
E L-Glutamic acid or D-Glutamic acid
K L-Lysine or D-Lysine
R L-Arginine or D-Arginine
P L-Proline or D-Proline
G Glycine

By way of a non-limiting example a constraint building block was prepared as follows.

Cysteamine (10 mmol) was dissolved in 20 ml trifluoroacetic acid. The solution was stirred at room temperature and a solution of acetamidomethanol (12 mmol) was added dropwise over a period of 30 minutes. The mixture was stirred for additional 120 minutes and the volatile parts removed in vacuo. The residue was dissolved in 80 ml water and the pH adjusted to 9. The product was then extracted with chloroform/isopropanol (3/1) and the solvents removed in vacuo. The crude product was then dissolved in a minimum of DCM and this solution added to a mixture of BOC-β-Ala (10 mmol), Cl—HOBt (10 mmol), DIEA (10 mmol) and DIC (20 mmol) in a minimum of DCM. After 12 hours the solution was washed with satured sodiumhydrogencarbonate, sodiumhydrogensulfate and sodiumchloride, dried over sodiumsulfate and the solvent removed in vacuo. The residue was dissolved in 10 ml trifluoroacetic acid and stirred for 60 minutes. Trifluoroacetic acid was then removed by coevaporation with DCM and the residue dissolved in DCM. The solution was neutralized by addition of DIEA and poured into a mixture of Fmoc-Glu-OtBu (10 mmol), Cl—HOBt (10 mmol), DIEA (10 mmol) and DIC (20 mmol). After 12 hours the solution was washed with satured sodiumhydrogencarbonate, sodiumhydrogensulfate and sodiumchloride, dried over sodiumsulfate and the solvent removed in vacuo. The residue was dissolved in 10 ml trifluoroacetic acid and stirred for 60 minutes. Trifluoroacetic acid then was removed by coevaporation with DCM and the residue dissolved in DCM. The crude constraint building block was purified by HPLC on a Kromasil C-18 column, eluted with acetonitrile-water gradient containing 0.1% v/v trifluoroacetic acid and lyophylized.

Another example of the synthesis of a building block is the following procedure:

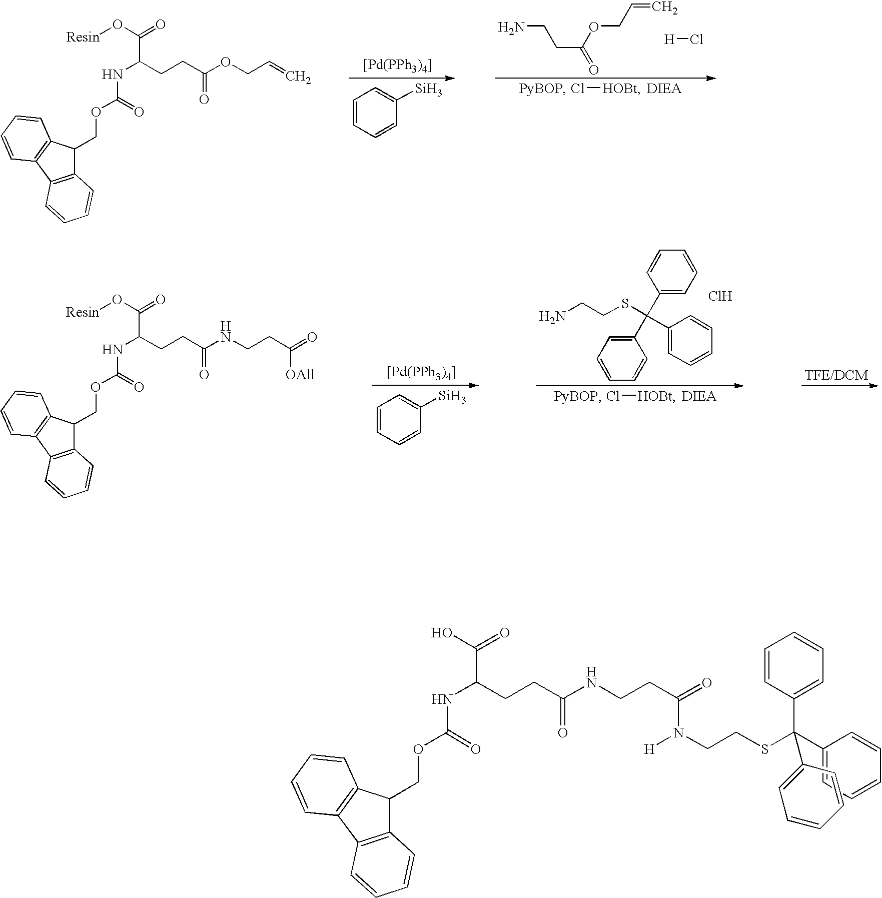

### 2-(9H-Fluoren-9-ylmethoxycarbonylamino)-4-[2-(2-tritylsulfanyl-ethylcarbamoyl)-ethylcarbamoy)-butyric acid

1. Loading of the Resin with N-α-Fmoc-L-glutamic Acid γ-allyl ester:

A solution of N-α-Fmoc-L-glutamic acid γ-allyl ester (20 mmol, 8.19 g) and diisopropylethylamine (DIEA) in a minimum of dry dichloromethane (DCM) is added to 10 g of 2-chlorotrityl resin (200-400 mesh, capacity 1.4 mmol/g). The mixture is shaken for 60 minutes. After removing of the reaction solution by filtration the resin is washed 3 times with each 80 ml of DCM/methanol/DIEA (80/15/5). 80 ml of DCM/MeOH/DIEA (80/15/5) are added to the resin which is shaken for 10 minutes and the solvent mixture is filtered off. This procedure is repeated once. The resin is washed 6 times with 200 ml DCM each.

In order to determine the loading of the resin with the amino acid the wet resin is weighed and an aliquot of about 100 mg is taken and dried at the air. The FMOC group is removed by DCM/piperidine and the amount of the piperidine/dibenzofulvene adduct determined by UV absorption at 330 nm. Typically, the loading is about 0.64 mmol/g dried resin.

2. Cleaving the allyl ester:

The resin is washed 3 times with 200 ml DCM each under argon. 200 ml dry DCM is added, argon is passed through the mixture for 15 minutes, 115 mmol phenyl silane (12.5 g) and 1 ml DIEA is added and argon is passed another 30 seconds through the mixture. 4.33 mmol Pd(PPh₃)₄ (5 g) (tetrakis(triphenylphosphine)palladium(0)) is added. After 3 hours the resin is washed 5 times with 200 ml DCM each, 5 times with 200 ml DMF each, once again washed 5 times with 200 ml DCM each, and 5 times with 200 ml DMF each.

3. Coupling of β-alanine allylester hydrochloride:

A solution of 19.5 mmol β-alanine allylester hydrochlorid (3.23 g), 25 mmol 6-chloro-1-hydroxybenztriazole (Cl—HOBt) (4.24 g), 75 mmol DIEA (12.39 ml) and 25 mmol PyBOP (13 g) (benzotriazole-1-yl-oxy-tris-pyrrolidino-phosphonium hexafluorophosphate) in 150 ml DMF is added to the deprocted resin. The mixture is shaken at room temperature ovemight. The resin is washed 6-times with 100 ml DMF each.

4. Cleaving the allyl ester

The resin is washed 3 times with 200 ml DCM each under argon. 200 ml dry DCM is added, argon is passed through the mixture for 15 minutes, 115 mmol phenyl silane (12.5 g) and 1 ml DIEA is added and argon is passed another 30 seconds through the mixture. 4.33 mmol Pd(PPh₃)₄ (5 g) (tetrakis(triphenylphosphine)palladium(0)) is added. After 3 hours the resin is washed 5 times with 200 ml DCM each, 5 times with 200 ml DMF each, once again washed 5 times with 200 ml DCM each, and 5 times with 200 ml DMF each.

5. Coupling of S-trityl-cysteamine:

A solution of 25 mmol Cl—HOBt (4.24 g), 25 mmol PyBOP (13 g) and 25 mmol DIEA (4.13 ml) is added to the deprotected resin in 75 ml DMF. After 1 Minute a solution of 25 mmol S-trityl-cysteamin-hydrochlorid (8.89 g) and 50 mmol DIEA (8.26 ml) in 75 ml DMF is added. The mixture is shaken overnight at room temperature. Afterwards the resin is washed 4 times with 100 ml DMF each and 3 times 100 ml DCM each.

6. Cleavage of the Product

The product is set free following successively these procedures:
(a) The resin is shaken for 2 hours with 200 ml 2,2,2-trifluoroethanol/DCM (50/50). The volatile compounds are removed in vaclio. DCM is added and removed in vacuo 3 times. Yield of crude product: 5.3 g (66%)
(b) The resin is shaken for 4.5 hours with 200 ml 2,2,2-trifluoroethanol/DCM (90/10). The volatile compounds are removed in vacuo. DCM is added and removed in vacuo 3 times. Yield of crude product: 0.5 g (6%)
(c) The resin is shaken for 3 days with 200 ml 2,2,2-trifluoroethanol/DCM (50/50). The volatile compounds are removed in vacuo. DCM is added and removed in vacuo 3 times. Yield of crude product: 0.73 g (9%)

Total Yield: 6.53 g (8.80 mmol, 82% according to initial resin loading) ¹H-NMR (400 MHz, DMSO-d₆), δ [ppm]: 7.93 (t, 1H, J=5.6 Hz), 7.89 (d, 2H, J=7.4 Hz), 7.82 (t, 1H, J=5.6 Hz), 7.72 (d, 2H, J=7.4 Hz), 7.66 (d, 1H, J=7.9 Hz), 7.41 (t, 2H, J-7.4 Hz), 7.36-7.20 (m, 17H), 4.30-4.15 (m, 3H), 3.95-3.89 (m, 1H), 3.23-3.14 (m, 2H), 2.99-2.92 (m, 2H), 2.24-2.10 (m, 6H), 2.00-1.90 (m, 1H), 1.80-1.68 (m, 1H), LC-ESI-MS: m/z=[M+H]: 743 (95%), [M+Na]: 765 (100%).

The sequences given in the examples below harbour target specific sequences, which can be used in the way described below, but might be modified without loss of desired action by means of single or multiple amino acid exchange operations. A substitution mutation of this sort can be made to change an amino acid in the resulting peptide in a non-conservative manner (i.e. by changing an amino acid belonging to a grouping of amino acids having a particular charge or size or other characterisitics to a grouping of amino acids with other grouping parameters) or in a conservative manner (i.e. by changing amino acids within one grouping of amino acids). Such a conservative change generally leads to less change in the structure and function of the resulting protein. A non-conservative change is more likely to alter the structure, activity or function of the resulting protein, although—if done at the right place—might be without deleterious effect on the target-interactions. The present invention should be considered to include sequences containing conservative and non-conservative changes, which do not significantly alter the activity or binding characteristics of the resulting modified peptide as compared to the original sequence. The following is one example of various groupings of amino acids:

Amino acids with nonpolar R Groups:
Alanine, Valine, Leucine, Isoleucine, Proline, Phenylalanine, Tryptophan, Methionine

Amino acids with uncharged polar R Groups:
Glycine, Threonine, Serine, Cysteine, Tyrosine, Asparagine, Glutamine

Amino Acids with charged polar R groups (negatively charged at pH 6):
Aspartic Acid and Glutamic Acid

Basic Amino Acids (positively charged at pH 6)
Lysine, Arginine, Histdine

Amino Acids with phenyl groups:
Phenylalanine, Tryptophan, Tyrosine

Particularly preferred conservative substitutions are: Lys for Arg and vice versa; Glu for Asp and vice versa; Ser for Thr and vice versa; Gin for Asn and vice versa.

Moreover, the invention includes modifications of the given binding regions of the peptide by amino acids, which transfer specific desired properties to the peptide. Such improvements include N- and/ot C-terminal modifications, which protect the peptides against exopeptidas cleavage. Preferred solutions of this problem include the use non-natural amino acids in terminal positions, especially preferred is the use of D-amino acids. Non-conservative exchanges inside the peptide sequence might be used to transfer better water solubility to non-binding but hydrophobic regions of the peptide. Chelating amino acids in N- or C-terminal postions can be use to enable the peptide to bind to metal-activated surfaces in order to assist purification and refolding during the production process.

### EXAMPLES

### Example 1

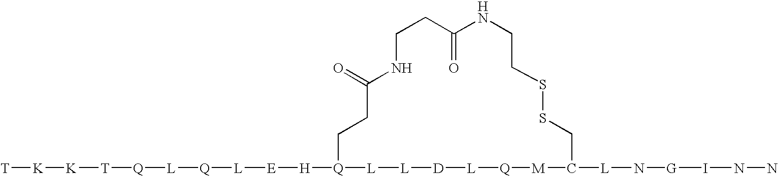

The bridge in example 1 connects the side chains of glutamine (glutamic acid respectively) and cysteine via beta-alanine and 2-aminoethanthiol. This compound represents an antagonist for the interleukin-2 receptor.

The last step of the synthesis of the cyclic helical constraint bridge is normally the formation of a disulfide bridge:

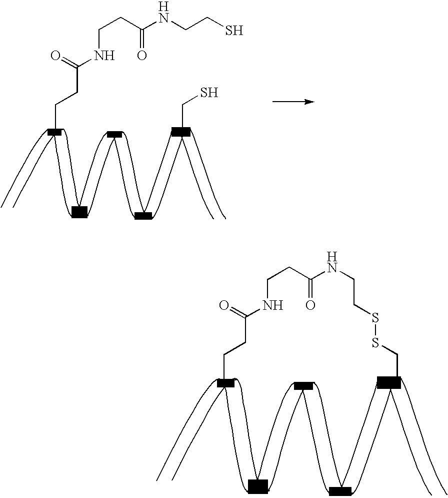

It has to be pointed out that this constraint bridge is custom-designed by molecular modelling. The bridge which connects the sidechains of amino acid i and i+7 has appropriate size and orientation to stabilize the helix without strain.

Furthermore, the bridge is stabilized by an aspartate side chain in position i+3 which acts as a supporting pillar. The hydrogen bond from one of the amide NH group to the aspartate side chain stabilizes the constraint and faciliates the synthesis of the bridge, because the correct conformation which leads to the formation of the disulfide bond is also stabilized.

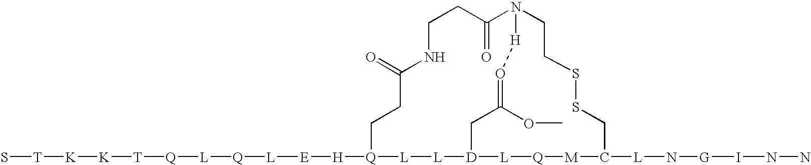

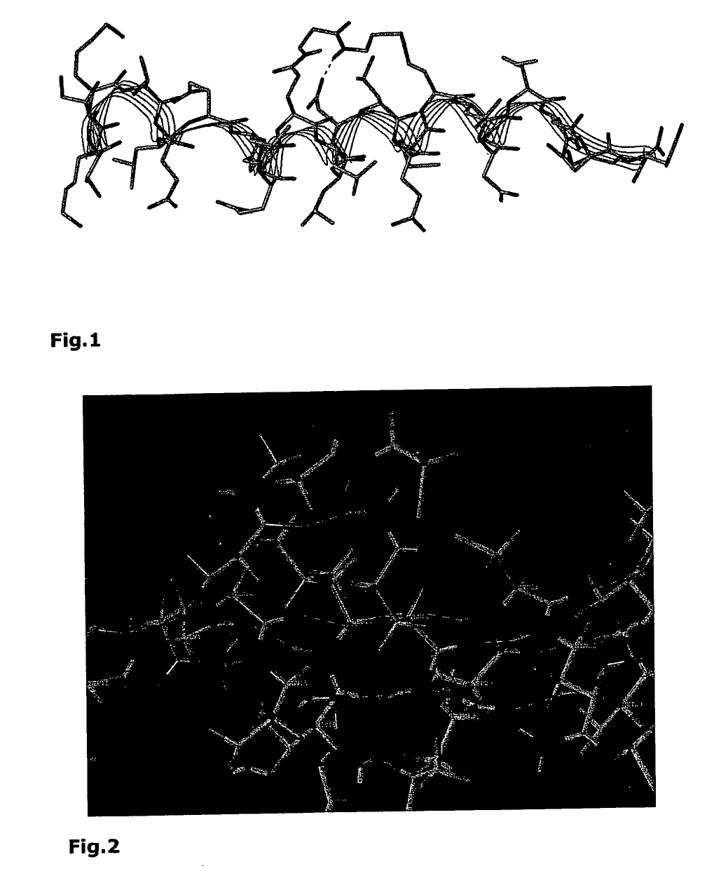

The three-dimensional molecular model in FIG. 1 demonstrates the stabilizing effect of the hydrogen bond to the aspartate side chain at position i+3. Thereby the invention provides a new way of stabilizing helical constraints by hydrogen bonds by amide structures in the bridge, combined with a disulfide bridge which is easy to form.

### Example 2

Another aspect in this invention is the stabilisation of the bridge from i to i+7 by a hydrogen bond from a glutamine side chain in position i+4. In this case, the supporting pillar is the hydrogen bond donor and the constraint bridge is the hydrogen bond acceptor. This is in contrast to the previous structure, where the supporting pillar was the hydrogen bond acceptor and the constraint bridge was the hydrogen bond donor. The respective three-dimensional model can be seen in FIG. 2.

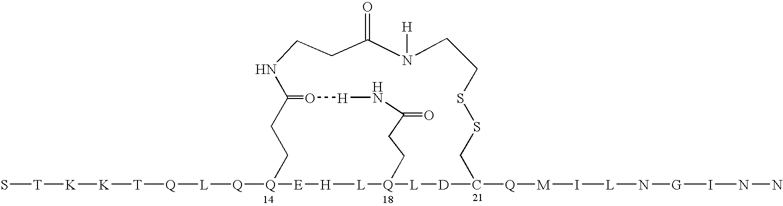

### Example 3

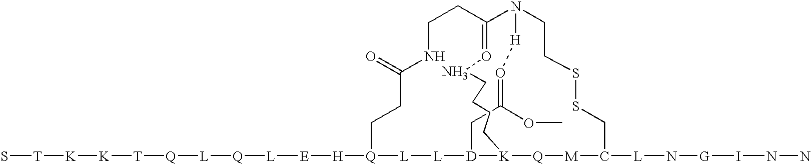

The constraint bridge from amino acid i to i+7 has appropriate size and orientation to stabilize the helix without strain.

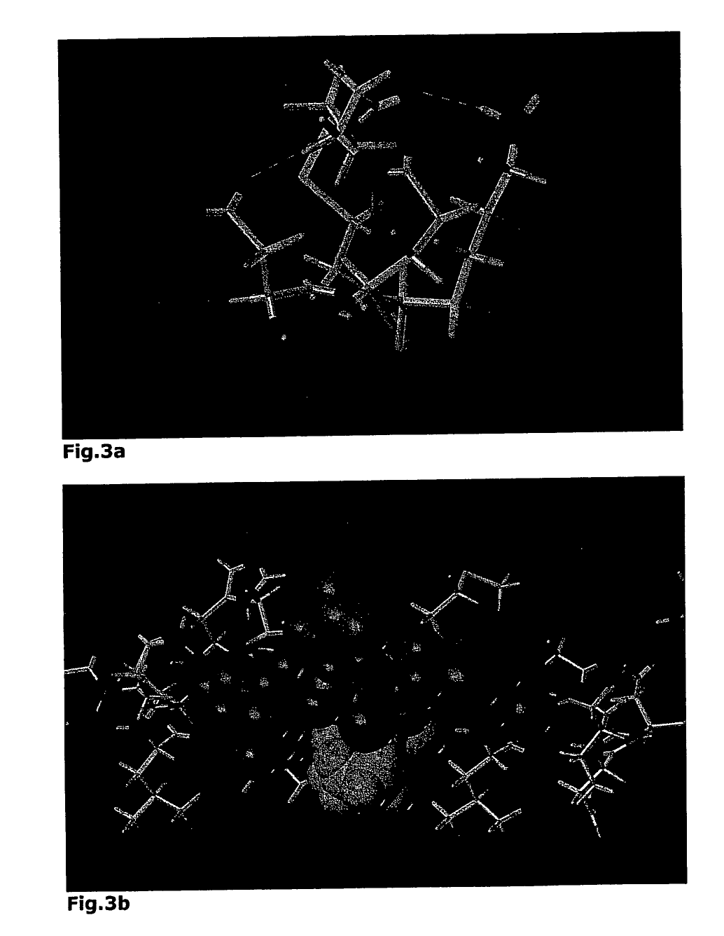

This bridge is stabilized by two custom-designed supporting pillars from two opposite sides represented by the sidechains of two standard amino acids. An aspartate side chain at position i+3 acts as a hydrogen-bond acceptor which connects to an amide NH group of the constraint bridge. Synchronously the constraint bridge is stabilized at the opposite side by a lysine side chain at position i+4 which acts as a hydrogen bond donor for a carbonyl group of the constraint bridge. The three-dimensional molecular models in FIGS. 3a and 3b demonstrate the stabilizing effect of the two supporting pillars from two opposite sides of the constraint bridge.

### Example 4

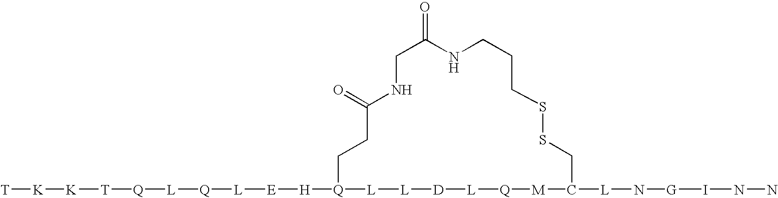

The bridge in example 4 connects the side chains of glutamine (glutamic acid respectively) and cysteine via glycine and 3-aminopropan-1-thiol. This compound represents an antagonist for the interleukin-2 receptor.

### Example 5

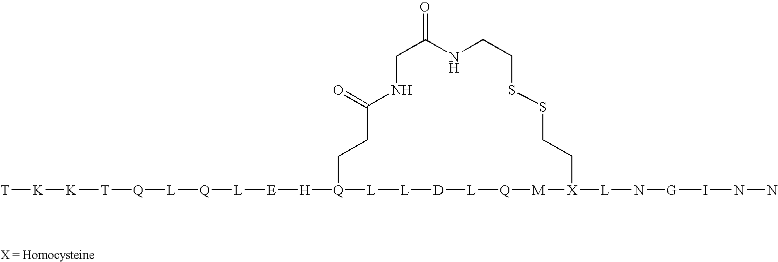

The bridge in example 5 connects the side chains of glutamine (glutamic acid respectively) and homocysteine via glycine and 2-aminoethanthiol. This compound represents an antagonist for the interleukin-2 receptor.

### Example 6

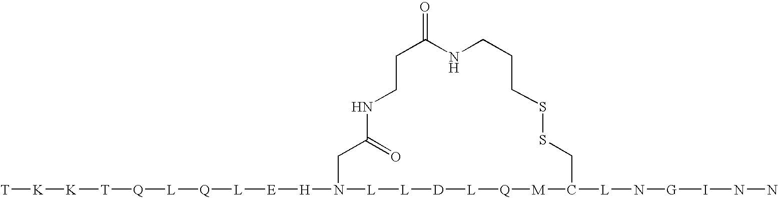

The bridge in example 6 connects the side chains of asparagine (aspartic acid respectively) and cysteine via beta-alanine and 3-aminopropan-1-thiol. This compound represents an antagonist for the interleukin-2 receptor.

### Example 7

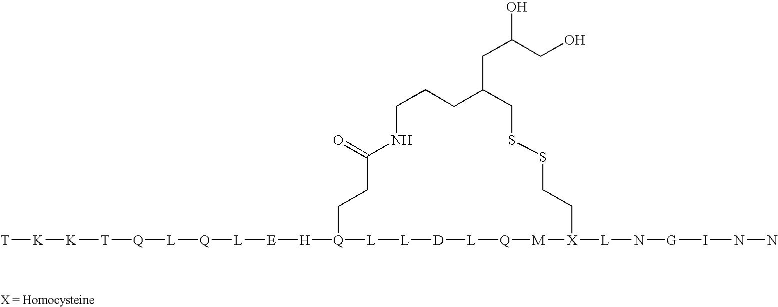

The bridge in example 7 connects the side chains of glutamine (glutamic acid respectively) and homocysteine via 5-aminopentari-1-thiol. The bridge backbone ist substituted with a sidechain containing two hydroxyl groups to improve the solubility of the compound.

This compound represents an antagonist for the interleukin-2 receptor.

### Example 8

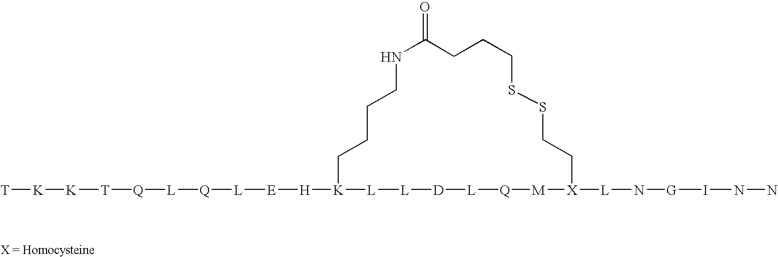

The bridge in example 8 connects the side chains of lysine and homocysteine via 3-thiopropionic acid. This compound represents an antagonist for the interleukin-2 receptor.

### Example 9

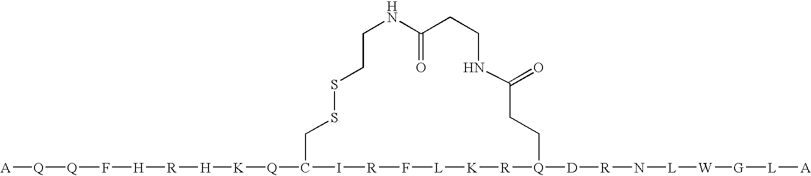

The bridge in example 9 connects the side chains of cysteine and glutamine (glutamic acid respectively) via beta-alanine and 2-aminoethanthiol. This compound represents an antagonist for the interleukin-4 receptor.

### Example 10

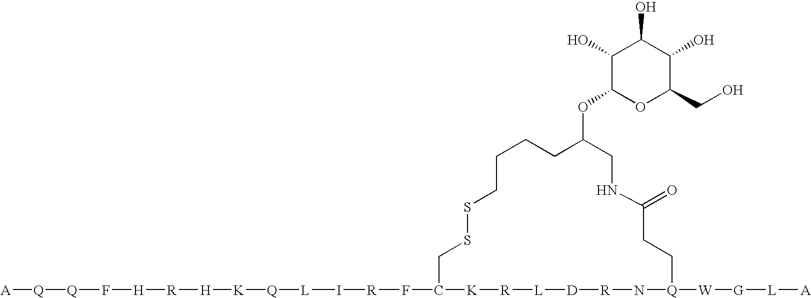

The bridge in example 10 connects the side chains of cysteine and glutamine (glutamic acid respectively) via omega-aminohexanthiol which is glycosylated to improve the pharmacokinetic properties of the compound. This compound represents an antagonist for the interleukin-4 receptor.

### Example 11

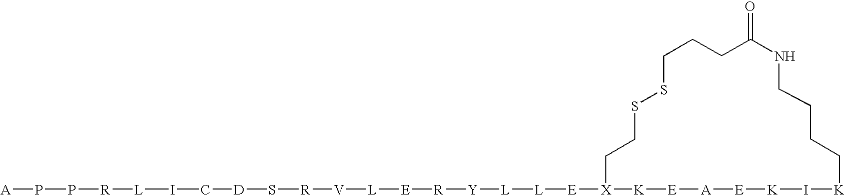

### Example 12

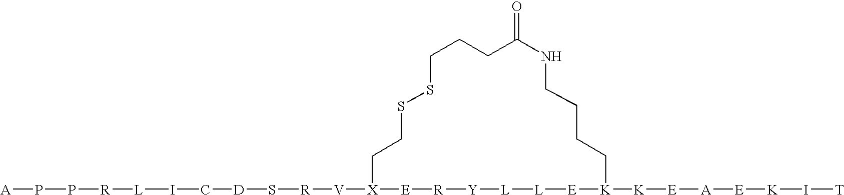

The bridges in example 11 and example 12 connect the side chains of homocysteine and lysine via 4-thiobutyric acid. This compounds represent binding molecules for the erythropoietin receptor.

### Example 13

Circular dichroism can be used to determine whether a peptide is helical or not. In a CD spectrum, a zero point at 200 nm and a minimum in “W” form between 200 and 250 nm are indications for a helical structure. Both criteria are independent of peptide concentration in solution.

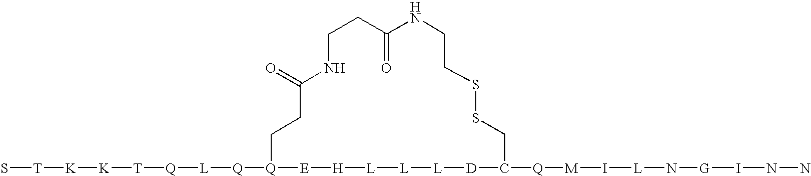

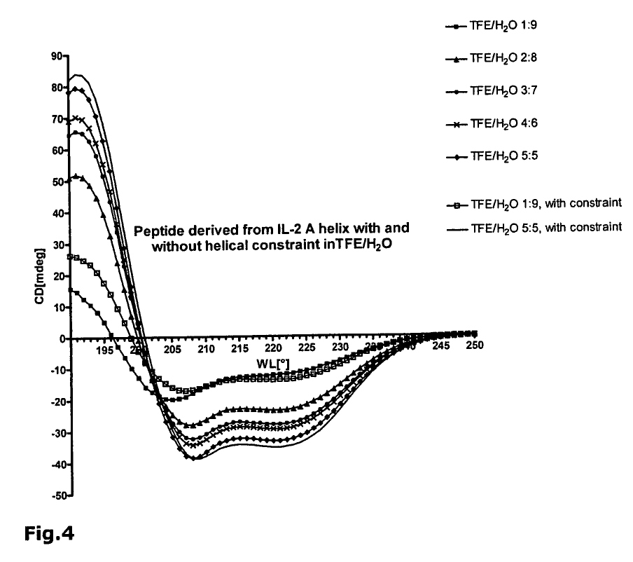

To find out how the helicity of the peptide in this example is influenced by trifluorethanol (TFE), the peptide with unclosed constraint was solved in TFE/H₂O 1:9, 2:8, 3:7, 4:6, and 5:5. Regarding FIG. 4 it can be noticed that zero points move to larger wave lengths and bands at ≈222 nm become more pronounced with increasing TFE concentration. From these facts it can be concluded that the amount of helical structure rises with increasing TFE concentration.

The peptide with closed helical constraint was solved in TFE/H₂O 1:9 and 5:5, respectively. In comparison to the spectra of peptides without helical constraint, zero points can be found at larger wave lengths and the band at 222 nm is stronger. Therefore it can be concluded that the helical constraint bridge attached to the peptide leads to a larger amount of helical structure.

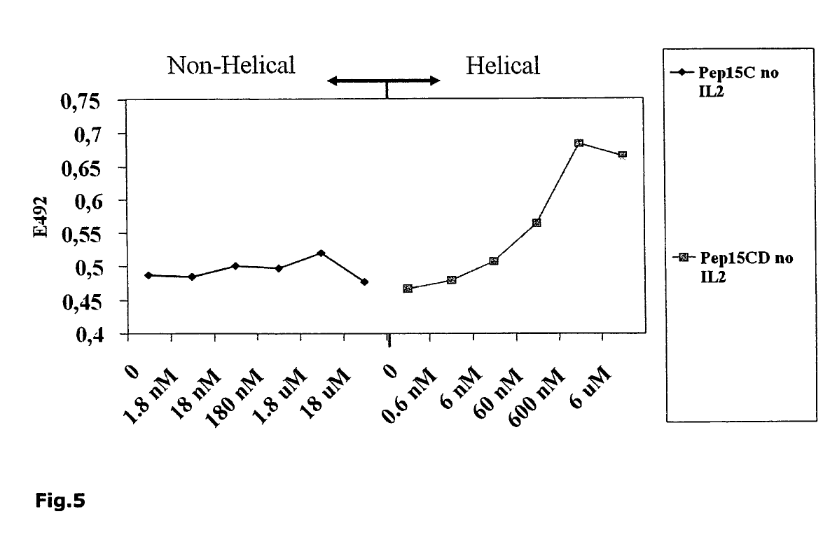

Helical content is a significant factor for biological effectivity of this peptide. FIG. 5 describes a NK-92 proliferation assay of the helical constrained IL-2R binding peptide described above (Pep15CD, right) in comparison with the corresponding native unconstrained peptide (Pep15C, left). The activity of the constrained helical peptide shows that the constraint bridge is effective and fixes the bioactive conformation of the peptide.

### REFERENCES

Braisted, A., K. J. Judice, et al. (1998). Constrained Helical Peptides and Methods of Making Same. T. Torchia. USA, Genentech, Inc.: 1-281.
Braisted, A. C., J. K Judice, et al. (1997). Constrained helical peptides and methods of making same. S. B. Piper Marbury Rudnick & Wolfe LLP; Kelber. USA, Genentech, Inc., South San Francisco, Calif.: 1-175.
Eckenberg, R., T. Rose, et al. (2000). The first alpha helix of interleukin (IL)-2 folds as a homotetramer, acts as an agonist of the IL-2 receptor beta chain, and induces lymphokine-activated killer cells.” Journal of Experimental Medicine 191(3): 529-540.
Gustchina, A., A. Zdanov, et al. (1995). “A model of the complex between interleukin-4 and its receptors.” Proteins: Structure, Function, and Genetics 21(2): 140-148.
Huston, J. S., L. L. Houston, et al. (1995). Biosynthetic binding proteins for immunotargeting. L. Testa Hurwitz & Thibeault. USA, Chiron Corporation, Emeryville, Calif. Creative BioMolecules, Inc., Hopkinton, Mass.: 1-30.
Jackson, D. Y., D. S. King, et al. (1991). “General Apprach to the Synthesis of Short a-Helical Peptides.” Journal of the American Chemical Society 113: 9391-9392.
Majewski (1996). “Interleukins.” Journal of Investigate Dermatology.
Phelan, J. C., N. J. Skelton, et al. (1997). “A General Method for Constraining Short Peptides to an a-Helical Conformation.” Journal of American Chemical Society 119(3): 454-460.
Somers, W., M. Stahl, et al. (1997). “1.9 A crystal structure of interleukin 6: implications for a novel mode of receptor dimerization and signaling.” EMBO Journal 16(5): 989-997.
Sung, S. S. and X. W. Wu (1996). “Molecular dynamics simulations of synthetic peptide folding.” Proteins: Structure, Function, and Genetics 25(2): 202-214.
Sytkowski, A. J. and J. Grodberg (1997). Recombinant human erythropoietin mutants. B. Hamilton, Smith & Reynolds, P.C. USA, Beth Israel Deaconess Medical Center, Boston, Mass.: 1-31.
Theze, J., R. Eckenberg, et al. (1999). Peptides of 11-2 and derivatives thereof and their use as therapeutic agents. B. Ores. Frankreich, Institut Pasteur, 25-28, rue du Docteur Roux, F-75724 Paris Cedex 15, France: 1-58.
Vieth, M., A. Kolinski, et al. (1994). “Prediction of the folding pathways and structure of the GCN4 leucine zipper.” Journal of Molecular Biology 237(4): 361-367.

1. Peptidic compounds having covalently closed bridge structures, which branch off from suitable amino acid side chains of a peptidic binding molecule with alpha-helical conformation and which connect at least two amino acid side chains of this peptide which are located at positions i and i+7 of the amino acid sequence of the peptide, thereby stabilizing the bridged part of the helix and being characterized by the presence of at least one amide (peptide) bond and at least one disulfide bridge, which both form part of the bridge backbone.

2. Peptidic compounds according to claim 1, wherein the bridge backbone ,including the side chain atoms of amino acids i and i+7 of the peptidic binding molecule, consists of one or two amide (peptide) bonds, one disulfide bridge and further 7 to 11, preferably 9 C- or N-atoms.

3. Peptidic compounds according to claim 2, wherein the bridge backbone comprises two amide (peptide) bonds, one sulfide bridge and further 7 carbon atoms.

4. Peptidic compounds according to claims 1 to 3, wherein the bridge is stabilized by one or more amino acid side chain(s) of the peptidic binding molecule by hydrogen bonds and the stabilizing amino acid(s) have a position between the two braching points of the bridge.

5. Peptidic compounds according to claim 4, wherein the stabilizing amino acid(s) are selected from lysine, arginine, arparagine, glutamine, aspartic acid, glutamic acid, serine, threonine, thyrosine, or histidine.

6. Peptidic compounds according to claim 5, wherein the stabilizing amino acid(s) is/are located at position(s) i+3 and/or i+4 of the peptides.

7. Peptidic compounds according to claim 6, wherein the stabilizing amino acid(s) is/are aspartate at position i+3, and/or lysine or glutamine at position i+4

8. Peptides compounds according to claims 1-7, and represented by the molecules covered by) one of the formulas (1a)-(1d): 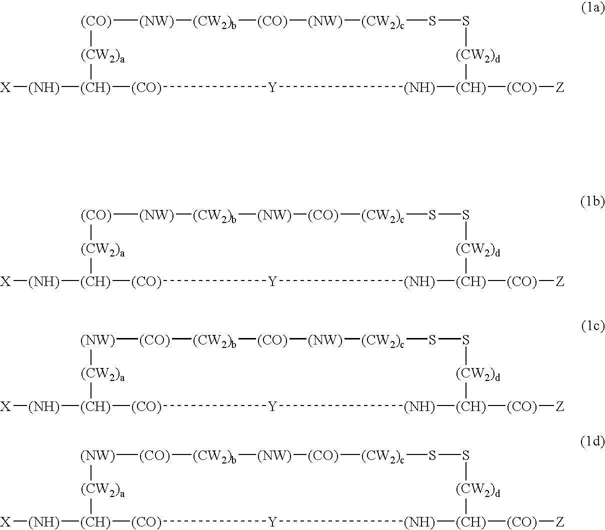
wherein X is hydrogen or any amino acid or any peptide or any compound represented by formula (1), (2) or (3), Y is any amino acid sequence consisting of six amino acids, Z is hydroxyl or any amino acid or any peptide or any compound represented by formula (1), (2) or (3); a, b, c and d are independently selected from the integers 1 to 3, provided that the sum a+b+c+d is 7, at each independent position of W, W can be freely chosen from hydrogen, a hydroxyl-, carboxyl- or amino group, an alkyl moiety with at least one hydroxyl-, carboxyl- or amino group, a polyethyleneglycol moiety, or a naturally occurring or artificial sugar molecule, and the peptides can consist of natural and/or unnatural D- and/or L-amino acids.

9. Peptidic compounds according to claim 1-7, and represented by the molecules covered by the generic formula (2): 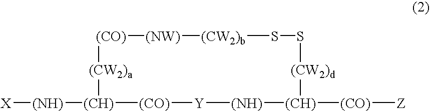
wherein X is hydrogen or any amino acid or any peptide or any compound represented by formula (1), (2) or (3), Y is any amino acid sequence consisting of six amino acids, Z is hydroxyl or any amino acid or any peptide or any compound represented by formula (1), (2) or (3), a, b and d are independently selected from the integers 1 to 5, provided that a+b+d is 9; at each independent position of W, W can be freely chosen from hydrogen, a hydroxyl-, carboxyl- or amino group, an alkyl moiety with at least one hydroxyl-, carboxyl- or amino group, a polyethyleneglycol moiety, or a naturally occurring or artificial sugar molecule, and the peptides can consist of natural and/or unnatural D- and/or L-amino acids.

10. Peptidic compounds according to claim 1-7, and represented by the molecules covered by the generic formula (3): 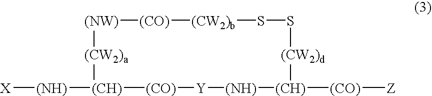
wherein X is hydrogen or any amino acid or any peptide or any compound represented by formula (1), (2) or (3), Y is any amino acid sequence consisting of six amino acids, Z is hydroxyl or any amino acid or any peptide or any compound represented by formula (1), (2) or (3), a, b and d are independently selected from the integers 1 to 5, provided that a+b+d is 9, at each independent position of W, W can be freely chosen from hydrogen, a hydroxyl-, carboxyl- or amino group, an alkyl moiety with at least one hydroxyl-, carboxyl- or amino group, a polyethyleneglycol moiety, or a naturally occurring or artificial sugar molecule, and the peptides can consist of natural and/or unnatural D- and/or L-amino acids.

11. Peptidic compounds according to claim 1-7, and represented by the molecules covered by one of the formulas (4a)-(4d): 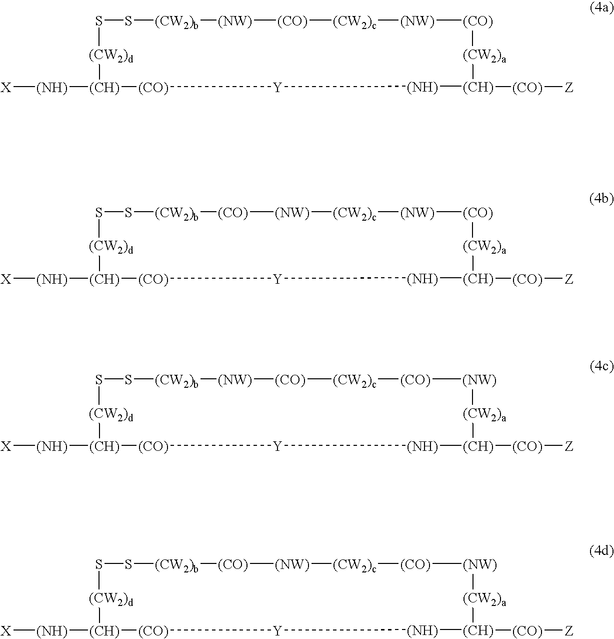
wherein X is hydrogen or any amino acid or any peptide or any compound represented by formula (1) to (2), Y is any amino acid sequence consisting of six amino acids, Z is hydroxyl or any amino acid or any peptide or any compound represented by formula (1) to (6), a, b, c and d are independently selected from the integers 1 to 3, provided that a+b+c+d is 7, at each independent position of W, W can be freely chosen from hydrogen, a hydroxyl-, carboxyl- or amino group, an alkyl moiety with at least one hydroxyl-, carboxyl- or amino group, a polyethyleneglycol moiety, or a naturally occurring or artificial sugar molecule, and the peptides can consist of natural and/or unnatural D- and/or L-amino acids.

12. Peptidic compounds according to claim 1-7, and represented by the molecules covered by the generic formula (5): 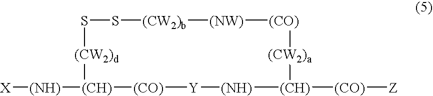
wherein X is hydrogen or any amino acid or any peptide or any compound represented by formula (1) to (6), Y is any amino acid sequence consisting of six amino acids, Z is hydroxyl or any amino acid or any peptide or any compound represented by formula (1) to (6), a, b and d are independently selected from the integers 1 to 5, provided that a+b+d is 9, W is hydrogen, a hydroxyl-, carboxyl- or amino group, an alkyl moiety with at least one hydroxyl-, carboxyl- or amino group, a peptide of maximally 30 amino acids, a polyethyleneglycol moiety, or a naturally occurring or artifical sugar molecule and the peptides can consist of natural and/or unnatural D- and/or L-amino acids.

13. Peptidic compounds according to claim 1-7, and represented by the molecules covered by the generic formula (6): 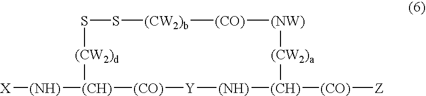
wherein X is hydrogen or any amino acid or any peptide or any compound represented by formula (1) to (6), Y is any amino acid sequence consisting of six amino acids, Z is hydroxyl or any amino acid or any peptide or any compound represented by formula (1) to (6), a, b and d are independently selected from the integers 1 to 5, provided that a+b+d is 9, at each independent position of W, W can be freely chosen from hydrogen, a hydroxyl-, carboxyl- or amino group, an alkyl moiety with at least one hydroxyl-, carboxyl- or amino group, a polyethyleneglycol moiety, or a naturally occurring or artificial sugar molecule, and the peptides can consist of natural and/or unnatural D- and/or L-amino acids.

14. Peptidic compounds according to claims 1-13, binding to the interleukin 2 receptor and containing the stabilized peptide sequence TKKTQLQLEHKLLDLQMXLNGINN in a helical conformation, where X stands for homocysteine and two helical turns are bridged by a backbone according to claims 1-13; thereby including non-exclusively the sequences and structures (a-f as follows:

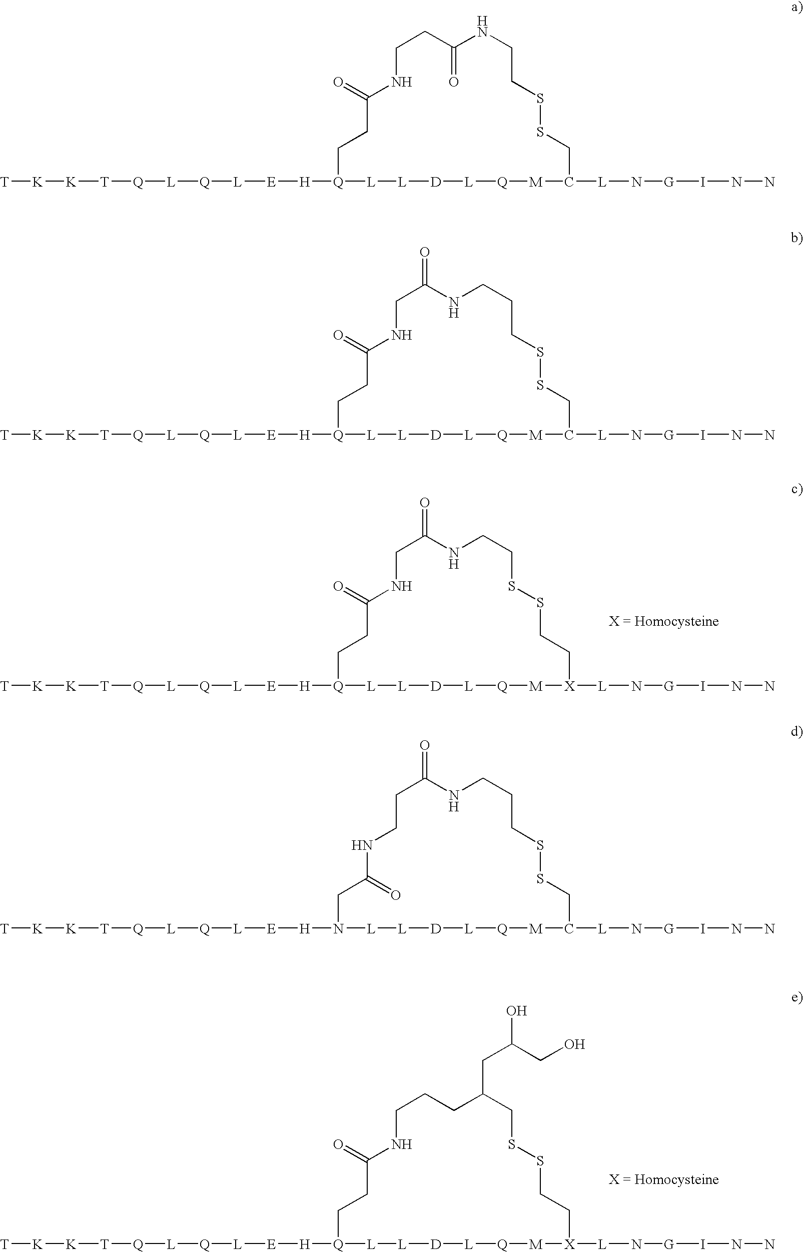

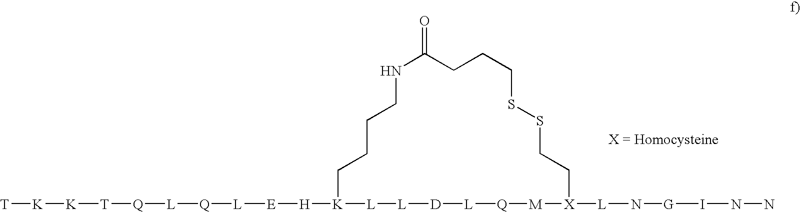

15. Peptidic compounds according to claim 14, in which the bridging structure is shifted along the peptide sequence in such a way that binding to the interleukin 2 receptor is maintained and another part of the overall helical structure is bridged by the construct.

16. Peptidic compounds according to claims 14 and 15, in which at least one amino acid of the peptide sequence is replaced by physicochemically related natural or non-natural amino acids in a conservative exchange, which maintains the binding of the peptide to the Interleukin 2 Receptor.

17. Peptidic compounds according to claims 14-16, which are N- and/or C-terminally modified in such a way that the binding of the peptide to the Interleukin 2 receptor is maintained and/or water solubility is improved and/or that exopeptidases can not cleave at the terminal sites, whereby terminal modifications include non-natural amino acids, D-amino acids, sugar moieties or freely chosen appropriate organic moieties.

18. Pharmaceutical preparations containing an active ingredient according to claims 14-17 and intended for use in humans or animals as an antagonist of the action of the cytokine Interleukin 2.

19. Peptidic compounds according to claims 1-13, binding to the interleukin 4 receptor and containing the stabilised peptide sequence AQQFHRHQCIRFLKRQDRNLWGLA in a helical conformation, wherein two helical turns are bridged by a backbone according to claims 1-13; thereby including nonexclusively the following sequence and structure (g):

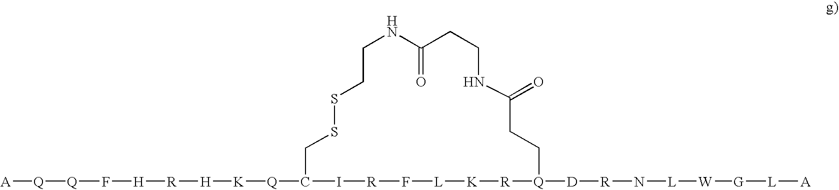

20. Peptidic compounds according to claim 19, in which the bridging structure is shifted along the peptide sequence in such a way that binding to the interleukin 4 receptor is maintained and another part of the overall helical structure is bridged by the construct.

21. Peptidic compounds according to claims 19-20, in which at least one amino acid of the peptide sequence is replaced by physicochemically related natural or non-natural amino acids in a conservative exchange, which maintains the binding of the peptide to the Interleukin 4 receptor.

22. Peptidic compounds according to claims 19-21, which are N- and/or C-terminally modified in such a way that the binding of the peptide to the Interleukin 4 receptor is maintained and/or water solubility is improved and/or that exopeptidases can not cleave at the terminal sites, whereby terminal modifications include non-natural amino acids, D-amino acids, sugar moieties or freely chosen appropriate organic moieties.

23. Pharmaceutical preparations containing an active ingredient according to claims 19-21 and intended for use in humans or animals as an antagonist of the action of the cytokine Interleukin 4.

24. Peptidic compounds according to claims 1-13, binding to the erythropoietin receptor and containing the stabilised peptide sequence APPRLICDSRVLERYLLEXKEAEKIK in a helical conformation, wherein two helical turns are bridged by a backbone according to claims 1-13; thereby including non-exclusively the following sequences and structures (h-i):

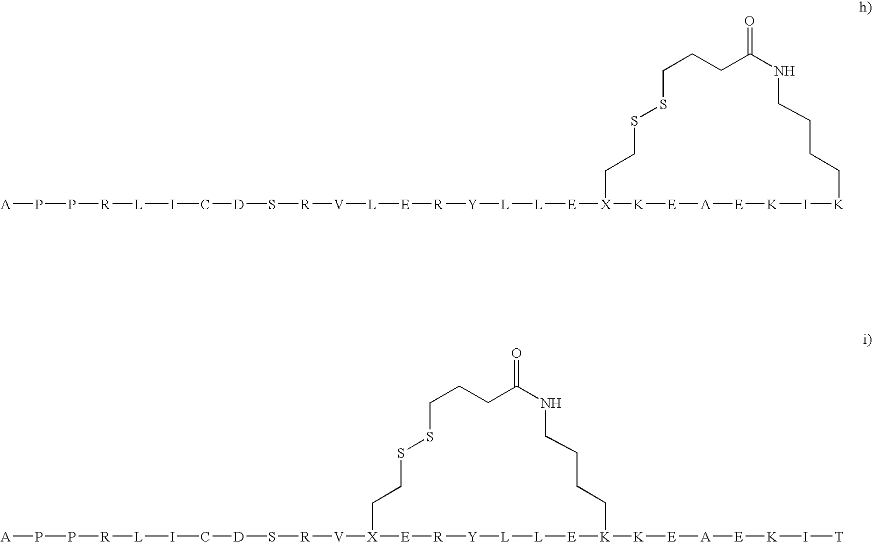

25. Peptidic compounds according to claim 24, in which the bridging structure is shifted along the peptide sequence in such a way that binding to the erythropoietin receptor is maintained and another part of the overall helical structure is bridged by the construct.

26. Peptidic compounds according to claims 24-25, in which at least one amino acid of the peptide sequence is replaced by physicochemically related natural or non-natural amino acids in a conservative exchange, which maintains the binding of the peptide to the erythropoietin receptor.

27. Peptidic compounds according to claims 24-26, which are N- and/or C-terminally modified in such a way that the binding of the peptide to the erythropoietin receptor is maintained and/or water solubility is improved and/or that exopeptidases can not cleave at the terminal sites, whereby terminal modifications include non-natural amino acids, D-amino acids, sugar moieties or freely chosen appropriate organic moieties.

28. Pharmaceutical preparations containing an active ingredient according to claims 19-22 and intended for use in humans or animals as an agonist of the action of the cytokine erythropoietin.

29. Mono- and polyclonal antibodies to the substances covered by claims 1-28, and the use of such antibodies in diagnostic and pharmacological quantification and/or inhibition of action of the active substances in body fluids or tissues of animals or humans.

30. Peptidic compounds according to claims 1-17, 19-22 and/or 24-27, in which the N-terminal amino acid is acetylated and/or the C-terminal amino acid is amidated.

31. Compounds as building blocks for the synthesis of peptidic compounds of any of claims 1-17, 19-22 and/or 24-27, represented by the molecules covered by the generic formulas (7a) to (7d): 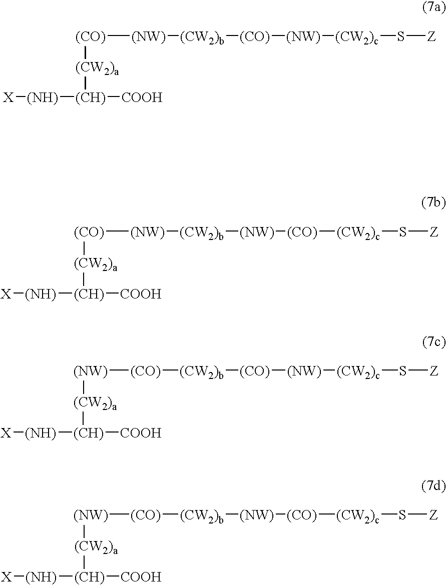
wherein X and Z are hydrogen or any protecting group; a, b, and c are independently selected from the integers 1 to 3, provided that the sum a+b+c is an integer from 3 to 6, at each independent position of W, W can be freely chosen from hydrogen, a hydroxyl-, carboxyl- or amino group, an alkyl moiety with at least one hydroxyl-, carboxyl- or amino group, a polyethyleneglycol moiety, or a naturally occurring or artificial sugar molecule.

32. Compounds as building blocks for synthesis of peptidic compounds of any of claims 1-17, 19-22 and/or 24-27, represented by one of the formulas (8a) to (8b): 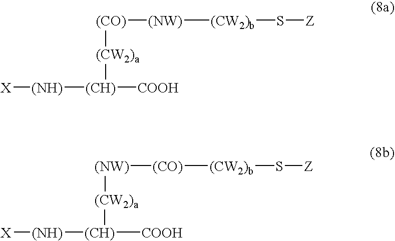
wherein X and Z are hydrogen or any protecting group; a and b are independently selected from the integers 1 to 5, provided that the sum a+b is an integer from 2 to 8, at each independent position of W, W can be freely chosen from hydrogen, a hydroxyl-, carboxyl- or amino group, an alkyl moiety with at least one hydroxyl-, carboxyl- or amino group, a polyethyleneglycol moiety, or a naturally occurring or artificial sugar molecule.

33. Compounds of claim 31 of the formula (9), wherein X and Z are hydrogen or any protecting group:

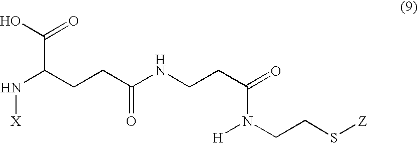

34. A compound of claim 33 of the formula (10):

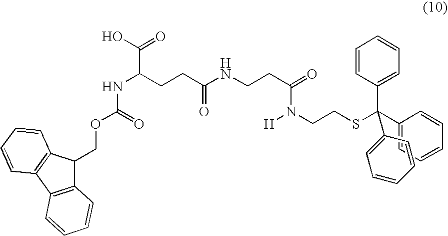

35. Methods for synthesis of building blocks according to claims 31 to 34 via solid phase synthesis.

36. Methods for synthesis of peptidic compounds according to claims 1-17, 19-22 and/or 24-27 comprising the following steps:
a. Synthesizing an intermediate peptidic compound by means of peptide synthesis from C- to N-term, comprising introduction of an amino acid containing a protected SH function in its sidechain at position i+7 (i.e. introduction after deprotection of the N-term of the amino acid at position i+8), followed by the introduction of six amino acids at positions i+6 to i+1, and furthermore followed by introduction of a building block according claims 31-34 at position i (i.e. after deprotection of the N-term of the amino acid at position i+1) of the growing peptide chain,
b. continuation of the peptide synthesis until the N-terminal amino acid was introduced
c. Removal of the remaining protecting groups,
d. establishing helix-stabilizing conditions, for example with appropriate fluorinated solvents,
e. obtaining the peptidic compound by closure of a disulfide bridge with appropriate reagents under these helix-stabilizing conditions.
